(19) United States
(12) Patent Application Publication
(10) Pub. No.: US 2024/0225808 A9
(43) Pub. Date: Jul. 11, 2024

(54) IMPROVEMENTS IN OR RELATING TO FLUID MANAGEMENT SYSTEMS

(71) Applicant: GREY MATTER DESIGNS LIMITED, London (GB)

(72) Inventor: William Howes, London (GB)

(73) Assignee: GREY MATTER DESIGNS LIMITED, London (GB)

(21) Appl. No.: 18/261,943

(22) PCT Filed: Jan. 14, 2022
(86) PCT No.: PCT/GB2022/050085

§ 371 Date: Jul. 18, 2023

(30) Foreign Application Priority Data
Jan. 18, 2021 (GB) 2100632.5

(51) Int. Cl. A61F 2/00

(52) U.S. Cl. CPC A61F 2/0018; A61F 2/0022; A61F 2210/009; A61F 2220/0008; A61F 2250/0069

(57) ABSTRACT

An implant for controlling fluid flow within a patient's urethra, the implant comprising a fluid pathway configured to receive a fluid; a seal configured to restrict fluid flow between the fluid pathway and the patient's urethra, in use, and a valve configured to control the flow of the fluid along the fluid pathway.

The present invention relates to fluid management systems and more specifically to the management of fluid in the urethra.

Incontinence is a health problem that adversely affects the quality of life of millions of sufferers around the globe. In the UK alone urologists estimate that urinary incontinence affects between 20 and 40% of the adult population. It is a health issue that has not seen great technological progress in the products available to treat the condition.

Loss of urinary continence can be caused by a range of factors including childbirth, spinal trauma, pelvic fracture and radical prostatectomy. Stress incontinence caused by the preceding factors is a common problem in both men and women and results in the involuntary and accidental passing of urine when pressure is applied to the bladder in coughing sneezing laughing or exercising. In these cases, the loss of urinary continence can be managed using catheterisation, absorbent products or other external devices.

Impaired bladder emptying can be caused by detrusor muscle failure, multiple sclerosis, spinal injury or benign prostatic hyperplasia (BPH) (an enlarged prostate gland) are conditions that can be managed using either long term indwelling catheters or intermittent catheterisation.

Management of incontinence or impaired bladder emptying by the use of catheters is common however both have attendant problems. Long term catheterisation requires a urine collection bag that has to be periodically emptied and is prone to blockages and urine leakage. This treatment is also the commonest source of nosocomial infection in the UK and accounts for a large number of urosepsis cases requiring hospital admission. Intermittent self-catheterisation is increasingly used with success but carries an incidence of infection as well as lifestyle restrictions in terms of finding suitable locations to carry out this sterile procedure. The use of long-term catheterisation and intermittent catheterisation both have significant lifestyle, social and economic impact.

Absorbent products are commonly employed to manage incontinence with sufferers having to regularly change and dispose of saturated pads or undergarments. While they are a convenient means of managing incontinence, skin sores and odour are unpleasant side effects for users alongside the ongoing financial and environmental costs associated with purchase and disposal of used pads.

It is against this background that the present invention has arisen.

According to the present invention there is provided an implant for controlling fluid flow within a patient's urethra, the implant comprising: a fluid pathway configured to receive a fluid, a seal configured to restrict fluid flow between the fluid pathway and the patient's urethra, in use, and a valve configured to control the flow of the fluid along the fluid pathway.

A seal that restricts fluid flow between the fluid pathway and the patient's urethra, in use, minimises the amount of fluid that can flow around the implant. This ensures that substantially all of the fluid flow from the bladder passes along the fluid pathway. The provision of a valve that is operable to control the flow of fluid along the pathway enables the patient to control the flow of urine from the bladder. This prevents fluid from constantly draining from the bladder, thus removing the need for a bag, container or absorbent pad to collect and store the fluid. Alternatively, or in addition, the valve may be configured to control the flow of the fluid into the fluid pathway.

The fluid pathway may be a conduit. The conduit may comprise at least one inner surface that defines the fluid pathway or at least a portion thereof.

The seal may be at least one outer surface of the conduit. Alternatively, or in addition, the seal may be at least one protrusion attached to an outer surface of the conduit. In some embodiments, the seal may be a sleeve configured to enclose the conduit and/or fluid pathway. The sleeve may be manufactured from silicon.

The implant may further comprise an expandable portion configured to secure the implant in a desired position, in use. The desired position may be at the vesico-urethural junction. Alternatively, the desired positon may be in the vicinity of the vesico-urethural junction. For example, the expandable portion may be located in the bladder. Alternatively, or in addition, the valve and/or seal may be positioned in the urethra.

In some embodiments, the desired position may be in the vicinity of the connection between the bladder and the urethra. Alternatively, the desired position may be the bladder neck, urethra neck and/or internal urethral orifice. For example, the expandable portion may be located within the bladder, and the seal may be located in the bladder neck, urethra neck and/or internal urethral orifice.

The expandable portion may be a malecot. The expandable portion may prevent the pressure of fluid in the bladder from expelling the implant back along the urethra.

The expandable portion may initially be in a collapsed state. In its collapsed state, the expandable portion may be sized to be inserted into the patient's bladder via the urethra. Consequently, in its collapsed state, the diameter of the expandable portion may be less than the diameter of the patient's urethra. Once in the desired position, the expandable portion may expand, thus preventing the implant from moving back along the urethra. The expandable portion will therefore ensure that the implant remains in the desired position.

The expandable portion may comprise a centroid and at least two deformable members configured to deform laterally away from the centroid, in use. At least two deformable members configured to deform laterally away the centroid of the expandable portion, in use, enable the implant to first be inserted along a patient's urethra before the members have deformed.

The deformable members may then be deformed to secure the implant in the desired position. The deformable members may prevent the implant from passing back along the urethra. Once the implant is in the desired position, the deformable members may be located in the patient's bladder. Advantageously, the use of at least two deformable members to secure the implant in a desired position allows fluid the flow between the deformable members, thus allowing the bladder to drain completely.

Alternatively, or in addition, the deformable members may be configured to deform laterally relative to a longitudinal axis of the patient's urethra, in use. More specifically, the at least two deformable members configured to buckle, in use.

The expandable portion may comprise at least three deformable members configured to buckle laterally away from the centroid, in use. At least three deformable members may provide at least three points of contact with the base of the bladder and/or neck of the urethra, once buckled, thus providing ample restraint against the pressure of fluid generated against the implant when the value is closed.

Each of the members may be a discrete portion, thus ensuring that fluid can flow between the members. In some embodiments, the expandable portion may comprise at least 2, 3, 4, 5, 6, 8, 10 or more than 10 deformable members. Each of the deformable members may be elongate. Alternatively, the expandable portion may comprise a single member configured to deform laterally relative to a longitudinal axis of the patent's urethra and/or a centroid of the expandable portion.

Each deformable member may comprise at least one groove configured to promote lateral deformation away from the centroid, in use. At least one groove may be used to provide a weak point within the deformable member, thus ensuring that member deforms laterally away from the centroid of the expandable portion, in use. This may prevent the expandable portion from ‘collapsing’ inward, thus reducing the likelihood of a blockage and increases the reliability of the implant.

Each groove may be located on a surface of the deformable member closest to the centroid of the expandable portion. Locating the groove on a surface of the deformable member that is closest to the centroid of the expandable portion ensures that each deformable portion buckles outwards and therefore laterally away from the longitudinal axis of the patient's urethra.

The implant may further comprise at least one recess. The at least one recess may be annular. The at least one recess may be located on the external surface of the fluid pathway. Alternatively, or in addition, the at least one recess may be located on the expandable portion. Accordingly, at least one of the external surfaces of the fluid pathway and the expandable portion may comprise at least one ridge. The at least one ridge may be annular. The ridge may be configured to move axially relative to the recess. The at least one ridge may be configured to engage with the at least one recess when the expandable portion is in its deformed position. Engagement of the at least one ridge with the at least one recess may prevent the expandable portion from returning to is undeformed position. There may be a plurality of ridges. There may be a plurality of recesses.

The fluid pathway may comprise an inlet positioned between the expandable portion and the seal. Positioning the inlet between the expandable portion and the seal means that the inlet may be located at the base of the bladder, or below, in use. This ensures that the bladder can drain completely. For example, the inlet may be located at the base of bladder, in use. Alternatively, or in addition, the inlet may be located within the bladder neck, urethra neck or internal urethral orifice, in use.

Consequently, at least a portion of the implant may be located in the patient's urethra. The portion of the implant located in the urethra may be a portion of the fluid pathway. Alternatively, the entire fluid pathway may be located in the urethra, in use.

The seal may comprise a plurality of protrusions attached to the outside of the fluid pathway. Alternatively, or in addition, the seal may comprise a plurality of protrusions attached to an outer surface of the fluid pathway. A plurality of protrusions restricts the amount of fluid that is able to flow around the implant in use. The protrusions may be substantially circular. Alternatively, or in addition, the protrusions and fluid pathway may be concentric.

At least one of the plurality of protrusions may comprise a diameter greater than the diameter of a bladder neck. Alternatively, at least one of the plurality of protrusions may comprise a diameter greater than the diameter of the patient's bladder neck. A protrusion with a diameter greater than the diameter of the bladder neck prevents the implant from entering the bladder. Alternatively, or in addition, at least one of the plurality of protrusions comprises a diameter greater than the diameter of the patient's urethra neck and/or internal urethral orifice.

The fluid flow rate in the urethra when the valve is in the closed position may be referred to as a leak rate. Alternatively, or in addition, the fluid flow rate through the seal and/or through the valve when the valve is in its closed position may be referred to as a leak rate. Accordingly, the leak rate may comprise the fluid flow rate through the seal and through the valve when the valve is in a closed position.

The implant may enable a leak rate in the urethra of less than 1 ml per hour when the valve is in a closed position. A leak rate of less than 1 ml per hour ensures that any fluid that passes through and/or around the implant when the valve is in the closed position is effectively unnoticeable by the patient. This prevents a receptacle or pad from being required to capture the ‘leaked’ fluid, which is of huge benefit to the patient. In some embodiments the leak rate in the urethra is less than 0.1 ml per hour, 0.25 ml per hour, 0.5 ml per hour, 1 ml per hour, 2 ml per hour, 5 ml per hour, 10 ml per hour, 20 ml per hour or 50 ml per hour when the valve is in the closed position. Alternatively, the leak rate in the urethra may be 0 ml per hour.

In some embodiments, the seal enables a leak rate of less than 1 ml per hour between the implant and the urethra, in use. Alternatively, the seal may enable a leak rate of less than 0.1 ml per hour, 0.25 ml per hour, 0.5 ml per hour, 1 ml per hour, 2 ml per hour, 5 ml per hour, 10 ml per hour, 20 ml per hour between or 50 ml per hour the implant and the urethra, in use. Alternatively, in some embodiments, the seal may enable a leak rate of 0 ml per hour. Consequently, the seal may entirely prevent fluid flow therethrough.

Alternatively, or in addition, the valve may enable a leak rate in the fluid pathway of less than 1 ml per hour when the valve is closed. Alternatively, the valve may enable a leak rate in the fluid pathway of less than 0.1 ml per hour, 0.25 ml per hour, 0.5 ml per hour, 1 ml per hour, 2 ml per hour, 5 ml per hour, 10 ml per hour, 20 ml per hour between or 50 ml per hour when the valve is closed. Alternatively, in some embodiments, the valve may enable a leak rate of 0 ml per hour when the valve is closed. Consequently, the valve may entirely prevent fluid flow therethrough when the valve is closed.

The implant may comprise a recess configured to provisionally engage with a delivery catheter. The recess configured to provisionally engage with a delivery catheter may be the second recess. Accordingly, the implant may comprise a first recess configured to engage with the ridge and prevent the expandable portion from returning to is undeformed position, and a second recess configured to provisionally engage with a delivery catheter. Alternatively, or in addition, the implant may comprise an aperture configured to provisionally engage with a delivery catheter. The recess and/or aperture may be shaped to provisionally engage with a delivery catheter. An implant comprising a recess and/or aperture configured to provisionally engage with a delivery catheter enables the implant to be inserted in the patient. The delivery catheter may then disengage with the implant, thus enabling the catheter to be removed and the implant to remain in the desired position within the patient.

The recess configured to provisionally engage with a delivery catheter may be shaped such that rotation of the delivery catheter, or a portion thereof, enables the implant to disengage from the catheter. Therefore, the recess may be shaped such that rotation of the delivery catheter, or a portion thereof, enables the implant to disengage from the catheter. The recess may comprise an opening, wherein the opening is positioned such that rotation of the delivery catheter, or a portion thereof, enables the implant to disengage from the catheter.

The valve may comprise at least one driven magnet configured to operate the valve between the open and closed position. A driven magnet configured to operate the valve between the open and closed position may be operated by a driving magnet. The driving magnet may be external to the implant. For example, the driving magnet may be external to the human body when the implant is in use. More specifically, the driving magnet may be held by the patient, wherein positioning the driving magnet in the vicinity of the driven magnet causes the valve to open.

Removing the driving magnet from the vicinity of the driven magnet may then cause the valve to close. Consequently, the valve may be biased towards the closed position. The bias may be generated by a bias magnet. Alternatively, the bias may be generated by a spring or by gravity. The bias may be overcome by the driving magnet.

Allowing a patient to operate the valve via a remote driving magnet ensures that the patient is in control of fluid flow through the implant.

The driven magnet may be operable in a direction substantially transverse to the direction of fluid flow in the fluid pathway. A driven magnet that is operable in a direction substantially transverse to the direction of fluid flow in the fluid pathway enables a driving magnet to more easily adjust the position of the driven magnet. This arrangement therefore allows for a stronger bias to be used within the valve as the stronger driven magnet can be more easily moved in the transverse direction. Advantageously, a stronger bias enables a more secure and/or more fluid tight seal to be generated within the valve.

The driven magnet may be coupled to a rotatable armature comprising a seal. The armature seal may be located within the fluid pathway. Alternatively, or in addition, the armature seal may be located externally with respect to the fluid pathway. The armature seal may be located at the entrance to the fluid pathway. For example, the armature seal may be located at the inlet. Rotation of the armature may operate the valve between the open and closed positions. Consequently, the armature may cause the seal to either allow or prevent the flow of fluid in the fluid pathway, thus opening or closing the valve. The use of an armature comprising a seal may enable a more fluid tight seal to be generated than using the magnet itself to generate a seal.

The valve may comprise at least two opposing driven magnets coupled to a rotatable armature. The armature may be configured to rotate about a pin located between the two driven magnets. The driven magnets may be arranged to generate opposing magnetic fields. Locating the centre of rotation in between two driven magnets reduces how far the armature is required to protrude outside the valve volume envelope. This decreases the overall size of the implant. Furthermore, the use of two opposing driven magnets reduces the strength required by the driving magnet to open the valve without needing to increase the size of the driven magnet.

The pin may be substantially equidistant between the two driven magnets. Positioning the pin substantially equidistant between the two driven magnets minimises how far the armature is required to protrude outside the valve volume envelope. This optimises the overall size of the implant.

The implant may comprise a bias magnet configured to hold the valve in a closed position. A bias magnet configured to hold the valve in a closed position ensures that the valve is not accidentally left open. The bias magnet may be positioned to attract at least one of the driven magnets such that the valve remains closed in the absence of driving magnet.

Moreover, according to the present invention there is also provided a deployment device for inserting an implant into a patient, the deployment device comprising a delivery catheter having a guide wire therein, wherein the guide wire comprises a distal end for attachment to the implant and a proximal end operably attached to a spring; and a trigger configured to hold the spring in tension and torsion, wherein actuation of the trigger releases the spring, thus generating axial and rotational movement of the guide wire to disconnect the implant from the guide wire, in use.

A deployment device operably connected to the implant and configured to be actuated by a trigger enables the implant to be inserted into a patient with ease. Moreover, the deployment device may remove the need for the implant to be touched by a human, thus retaining sterility.

The deployment device may comprise a cam located between the spring and the guide wire. The cam may be configured to transmit axial movement from the spring to the guide wire followed by rotational movement from the spring to the guide wire followed by axial movement from the spring to the guide wire following actuation of the trigger.

For example, the cam may comprise three channels, wherein the second channel is substantially perpendicular to the first and third channels. The three channels may be connected to form a single channel comprising three portions. The three channels may be shaped to generate axial movement, followed by rotational movement, followed by axial movement of the cam, thus guide wire, following actuation of the trigger.

The first axial movement of the guide wire may cause the expandable portion to expand. The rotational movement may cause the guide wire to disengage with the implant. The second axial movement may cause the guide wire to retract from implant, thus enabling safe removal of the delivery catheter from the patient.

The delivery catheter may comprise a protrusion configured to be provisionally received by a recess in the implant. Alternatively, the delivery catheter may comprise a protrusion configured to be provisionally received by an aperture in the implant. The protrusion enables a secure connection to be initially made between the implant and the deployment device. Rotational movement of the guide wire may cause rotational movement of the protrusion, thus disconnecting the implant from the deployment device.

The guide wire may be operably attached to the distal end of the expandable portion of the implant. Consequently, actuation of the trigger may cause deformation of the expandable portion of the implant. Alternatively, or in addition, the first axial movement of the guide wire may cause the expandable portion of the implant to expand. For example, in use, actuation of the trigger may release the spring, thus ‘pulling’ the distal end of the expandable portion towards the proximal end of the expandable portion and causing the deformable members to deform laterally away from the centroid of the expandable portion.

The implant may further comprise a cord. The cord may be connected to the fluid pathway. At least a portion of the cord may remain outside the patient's body when the implant is in use. The implant may be removed by pulling on the cord. Pulling on the cord may cause the expandable portion to return to its undeformed position. The cord may be a suture. Alternatively, the cord may be a monofilament.

The invention will now be further and more particularly described, by way of example only, with reference to the accompanying drawings.

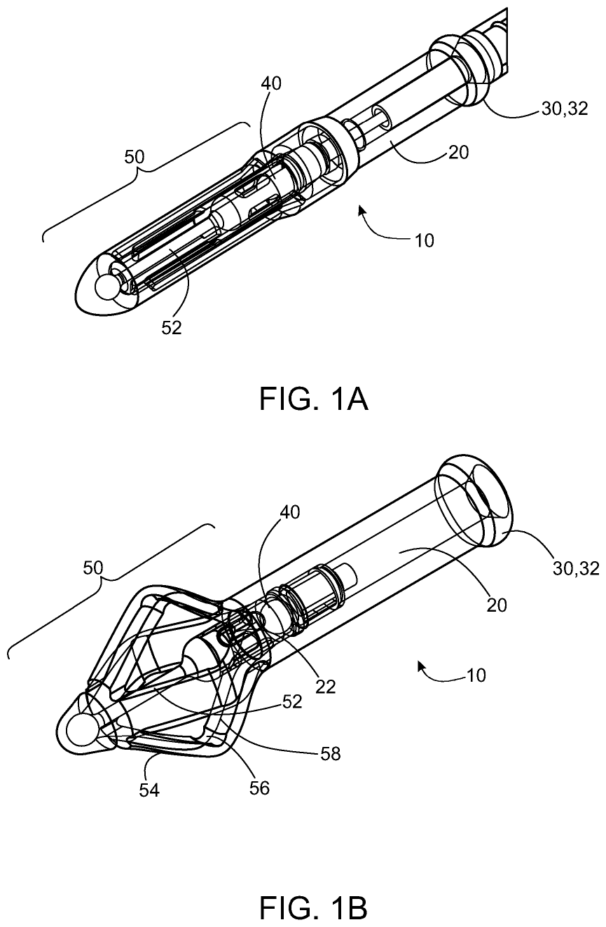

FIG. 1A shows an implant in a collapsed position;

FIG. 1B shows the implant of FIG. 1A in an expanded position;

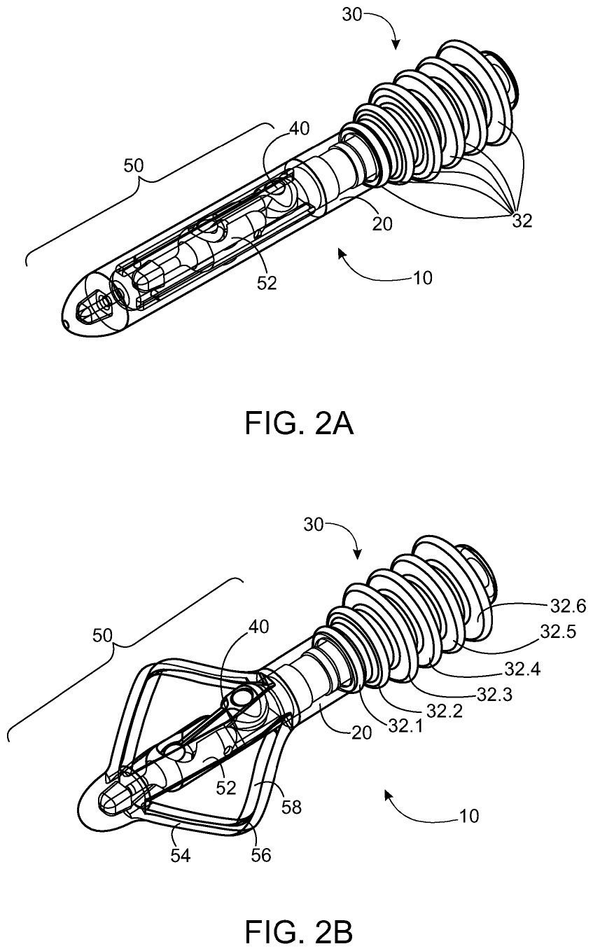

FIG. 2A shows an implant in a collapsed position comprising an external seal;

FIG. 2B shows the implant of FIG. 2A in an expanded position;

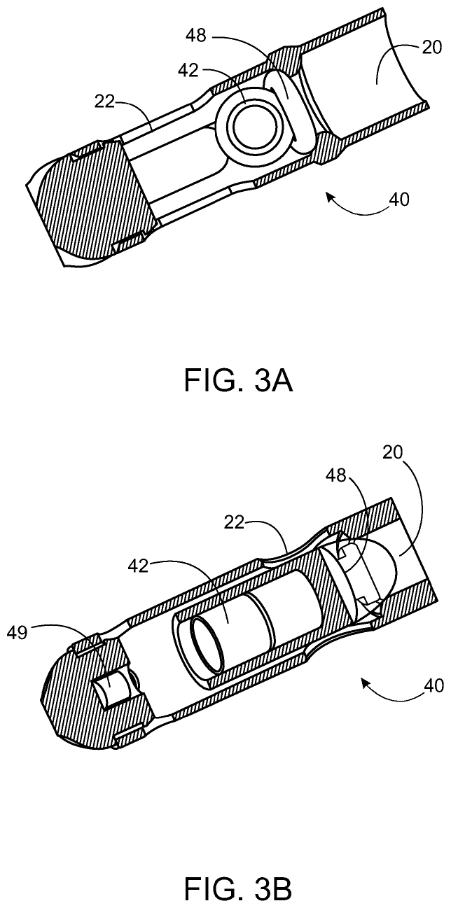

FIG. 3A shows a valve comprising a ball seal;

FIG. 3B shows a valve comprising a piston seal;

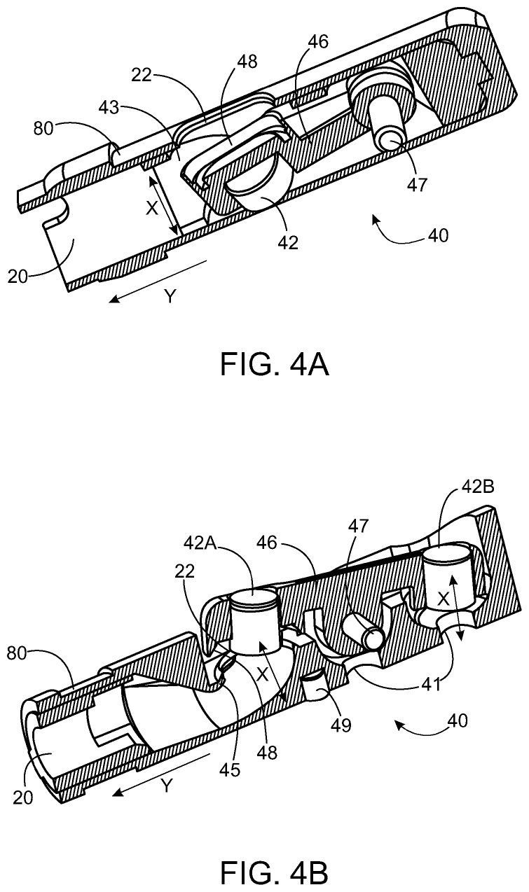

FIG. 4A shows a valve comprising rotatable armature;

FIG. 4B shows a valve comprising a rotatable armature and two driven magnets;

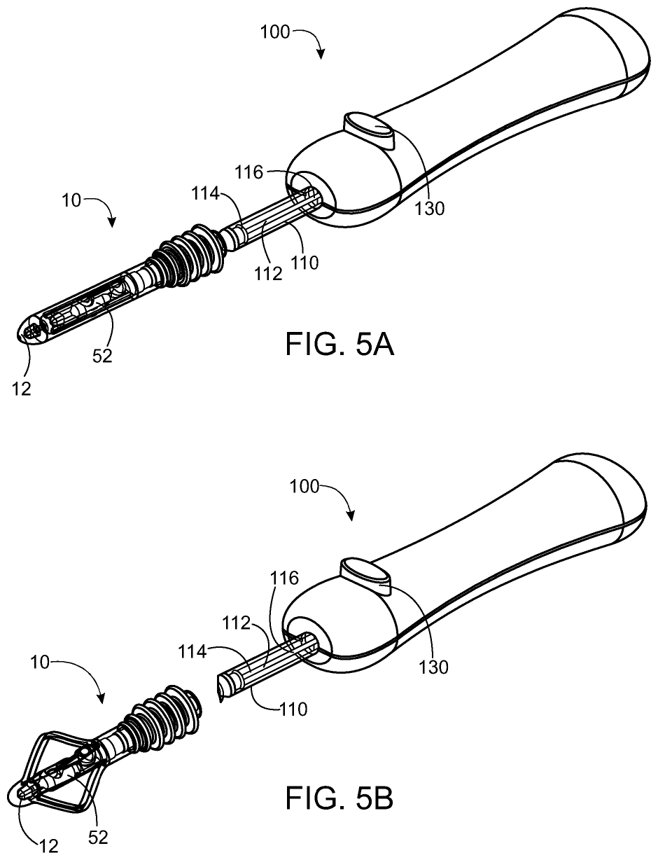

FIG. 5A shows the implant and deployment device before actuation;

FIG. 5B shows the implant and deployment device of FIG. 5A following actuation;

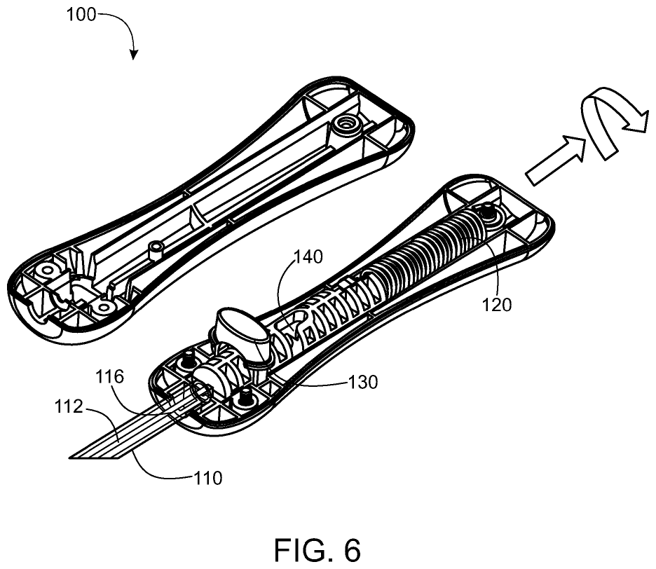

FIG. 6 shows the deployment device;

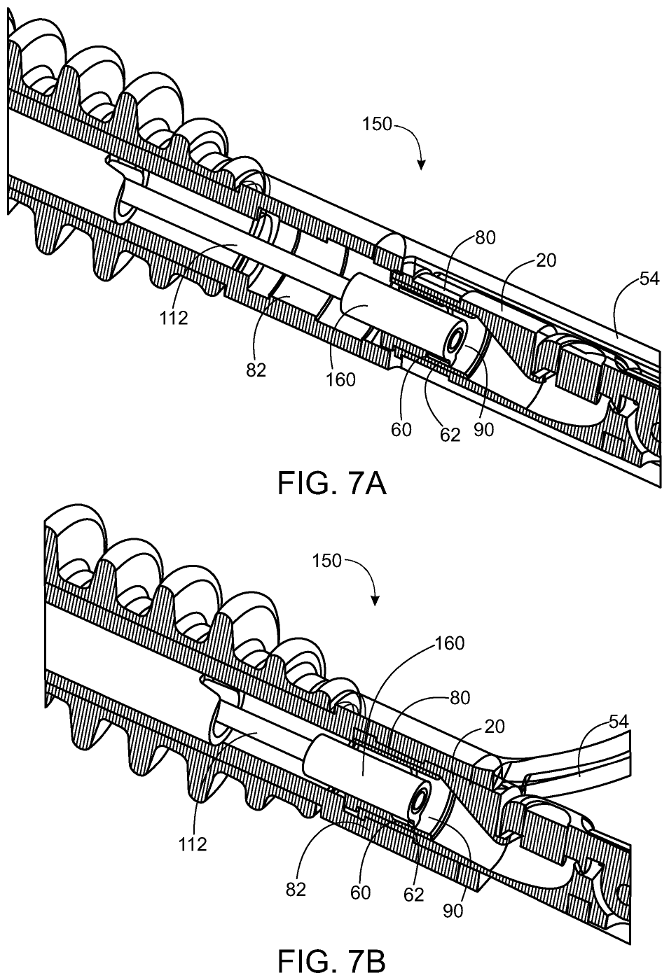

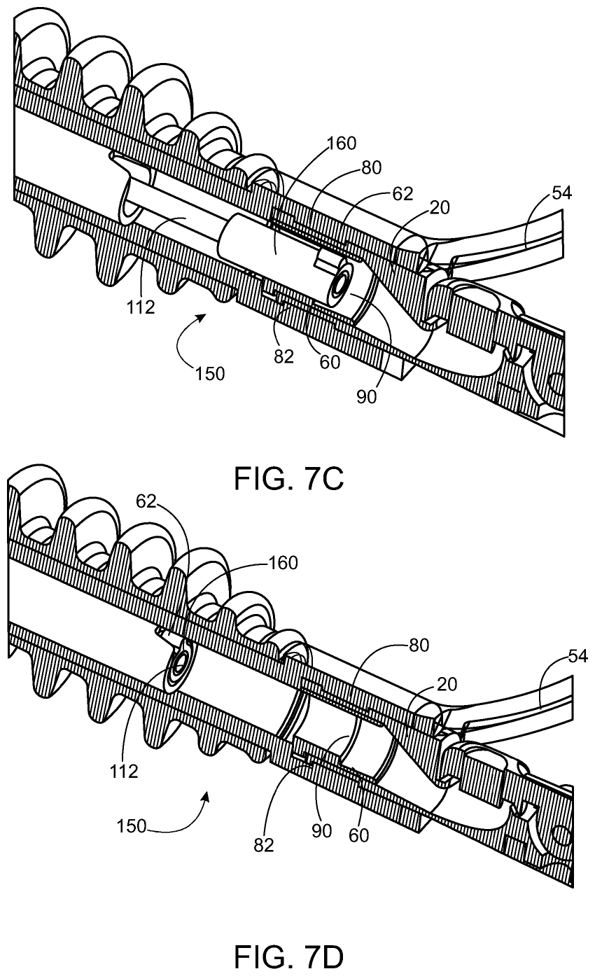

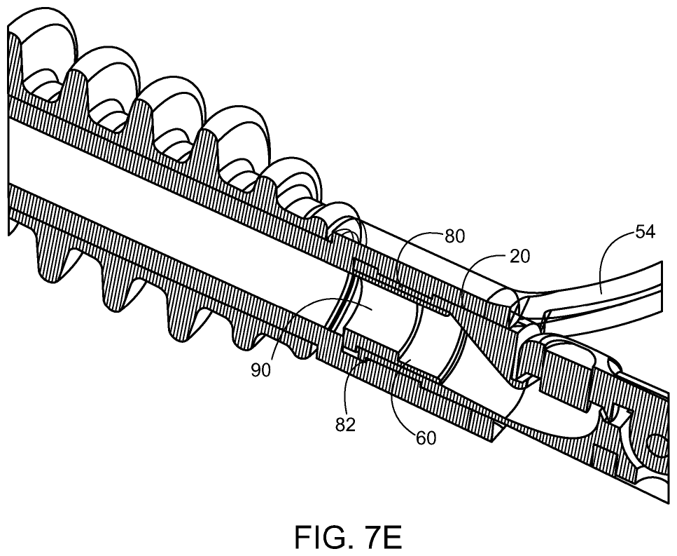

FIG. 7A shows the connection between the implant and the deployment device;

FIG. 7B shows the connection between the implant and the delivery catheter when the expandable portion has deformed;

FIG. 7C shows the implant when the delivery catheter has disengaged;

FIG. 7D shows the implant when the delivery catheter is being retracted;

FIG. 7E shows the implant in situ;

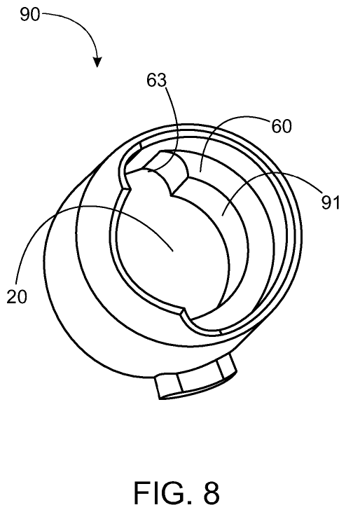

FIG. 8 shows the seat configured to provisionally connect the implant to the guide wire connector.

FIGS. 1A to 2B show an implant 10 for controlling fluid flow from a patient's bladder and through a patient's urethra. The implant 10 comprises a fluid pathway 20, a seal 30 and a valve 40. The implant 10 further comprises an expandable portion 50. FIGS. 1A and 2A shows the expandable portion 50 in an unexpanded configuration whereas FIGS. 1B and 2B show the expandable portion 50 in an expanded configuration. When expanded, the expandable portion 50 is configured to secure the implant 10 in a desired position, such as the neck of the bladder, in use.

The seal 30 is configured to restrict fluid flow between the fluid pathway 20 and the patient's urethra, in use. This ensures that substantially all of the fluid within a patient's bladder enters the urethra via the fluid pathway 20. In FIGS. 1A and 1B, the seal 30 is a portion of an external wall of the fluid pathway. For example, in some embodiments, the fluid pathway 20 is a conduit and the seal 30 is a portion of an external wall of the conduit. The outer diameter of the conduit is approximately equal to the diameter of the patient's bladder neck. The bladder neck is the thinnest point between the bladder and the urethra and the therefore the implant 10 is able to be inserted along the urethra before forming a substantially fluid tight seal in the bladder neck.

In FIGS. 2A and 2B, the seal 30 comprises a plurality of protrusions attached to the outer surface of the fluid pathway 20. In some embodiments, each protrusion is of substantially equal size and/or diameter. For example, in some embodiments, each protrusion may be approximately 15 F to 45 F in diameter. Alternatively, each protrusion may be approximately 5 mm to 15 mm in diameter.

However, in FIGS. 2A and 2B, the seal 30 comprise a plurality of different sized protrusions. For example, the first protrusion 32.1 has the smallest in diameter and last protrusion 32.6 has the largest in diameter. For example, in some embodiments, the first protrusion 32.1 is approximately 18 F to 24 F and the last protrusion 32.6 is approximately 27 F to 33 F. Alternatively, in some embodiments, the first protrusion 32.1 is approximately 6 mm to 8 mm and the last protrusion 32.6 is approximately 9 mm to 11 mm. The second protrusion 32.2 is approximately is approximately 18 F to 28 F, or 6 mm to 10 mm. The third, fourth and fifth protrusions 32.3, 32.4, 32.5 have a substantial equal diameter sized between that of the first protrusions 32.1 and last protrusions 32.6. For example, in some embodiments, the third, fourth and fifth protrusions 32.3, 32.4, 32.5 are approximately 20 F to 30 F. Alternatively, in some embodiments, the third, fourth and fifth protrusions 32.3, 32.4, 32.5 are approximately 7 mm to 10 mm.

However, any number or protrusions 32 may be used. For example, in some embodiments, the seal comprises 1, 2, 3, 4, 5, 6, 7, 8, 9, 10 or more than 10 protrusions.

Moreover, each protrusion 32 is flexible and/or soft. Consequently, each protrusion 32 will collapse sideways when they pass through a lumen larger than the size of the fluid pathway but less than the diameter of the protrusion 32. For example, each protrusion 32 will collapse sideways when they pass through a urethra larger than the size of the fluid pathway but less than the diameter of the protrusion 32. When a protrusion 32 is caused to collapse, it collapses into the trough between the adjacent protrusions. An adjacent protrusion cannot prevent the collapsing protrusion from collapsing. The width of the trough after a protrusion is defined by the previous protrusion height.

The last protrusion 32.6 is the largest protrusion and comprises a diameter greater than the diameter of the bladder neck. Consequently, the last protrusion 32.6 acts as a backstop to reduce the risk of the implant migrating into the bladder.

The fluid pathway 20 is configured to receive a fluid via an inlet 22. In some embodiments, the fluid pathway 20 comprises an inlet 22 positioned between the expandable portion 50 and the seal 30. This ensures that the inlet 22 is located below the base of the bladder, in use, therefore enabling the bladder to drain entirely. The fluid pathway 20 is approximately 12 F to 24F, 15 F to 21 F or 18 F in diameter. Alternatively, in some embodiments, the fluid pathway 20 is approximately 4 mm to 8 mm, 5 mm to 7 mm or 6 mm in diameter.

The valve 40 is operable between an open position and closed position. When the valve is closed, the seal and the valve restrict the flow of fluid from the bladder to less than 0.25 ml per hour. More specifically, the valve enables a leak rate of less than 0.2 ml per hour and the seal enables a leak rate of less than 0.05 ml per hour. In some embodiments, the leak rate is approximately 0 ml per hour. When the valve is open, the valve may enable a fluid flow rate of 3 to 4 ml per second. However, the flow rate is driven by the head pressure within the bladder. Consequently, when the valve is open, the valve may enable a fluid flow rate of up to 0.5, 1, 2, 3, 4, 5, 6, 8, 10 or more than 10 ml per second.

FIGS. 1A and 2A shows the expandable portion 50 in an unexpanded configuration whereas FIGS. 1B and 2B show the expandable portion 50 in an expanded configuration. The expandable portion 50 comprises a centroid 52 and at least two deformable members 54 configured to deform laterally away from the centroid 52, in use.

In some embodiments, the expandable portion comprises a plurality of deformable portions. For example, in some embodiments the expandable portion comprises 1, 2, 3, 4, 5, 6, 7, 8, 9, 10 or more than 10 deformable members.

Each deformable member 54 comprises at least one groove 56 configured to promote lateral deformation away from the centroid 52, in use. Each groove 56 is located on a surface 58 of the deformable member 54 closest to the centroid 52 of the expandable portion 50. Therefore, in use, each deformable member 54 is configured to buckle laterally away the centroid 52.

The implant 10 further comprises a first recess 80. The recess 80 is annular. The recess 80 is located on the external surface of the fluid pathway 20. Furthermore, the implant 10 comprises an annular ridge 82. The annular ridge 82 is configured to engage with the annular recess 80 when the expandable portion 50 is in its deformed position. Engagement of the ridge 82 with the recess 80 prevents the expandable portion 50 from returning to is undeformed position.

FIG. 3A shows a valve 40 comprising a ball seal. The valve 40 comprises a driven magnet 42 in the form of a spherical ball that is configured to be moved by an external driving magnet. When the valve 40 is in a closed position, as shown in FIG. 3A, the driven magnet 42 is pressed into the seal 48, thus preventing fluid flow within the fluid pathway 20 downstream of the seal 48. Accordingly, the valve 40 may comprise a bias magnet, not shown, configured to hold the driven magnet 42 against the seal 48. In use, a patient may position the external driving magnet in the vicinity of the implant, thus forcing the driven magnet 42 away from the seal 48 and allowing fluid from the bladder to enter the fluid pathway 20 downstream of the seal 48.

FIG. 3B shows a valve 40 comprising a piston seal. The valve comprises a driven magnet 42 in the form of a piston that is configured to be moved by an external driving magnet. When the valve 40 is in a closed position, the driven magnet 42 is pressed into the seal 48, thus preventing fluid flow within the fluid pathway 20 downstream of the seal 48. Accordingly, the valve 40 may comprise a bias magnet 49 configured to hold the driven magnet 42 against the seal 48. In use, a patient may position the external driving magnet in the vicinity of the implant, thus forcing the driven magnet 42 away from the seal 48 and allowing fluid from the bladder to enter the fluid pathway 20 downstream of the seal 48, as shown in FIG. 3B.

FIG. 4A shows a valve 40 comprising rotatable armature 46. The valve comprises a driven magnet 42 attached to an armature 46 that is configured to rotate about a pin 47. The driven magnet 42 is operable in a direction, X, which is substantially transverse the direction of fluid flow, Y, in the fluid pathway, as indicated in FIG. 4A.

The armature 46 comprises a seal 48 configured to generate a substantially fluid tight seal against an inner surface of the fluid pathway 20. More specifically, the seal 48 is configured to generate a substantially fluid tight seal against a ridge 43 on the inner surface of the fluid pathway 20. Rotation of the armature 46 is caused when an external driving magnet is positioned in the vicinity of the implant 10. When the valve 40 is in a closed position, the seal 48 is pressed against the ridge 43, thus preventing fluid flow into the fluid pathway 20 downstream of the seal 48. Accordingly, the valve 40 may comprise a bias magnet, not shown, configured to hold the driven magnet 42 against the ridge 43. In use, a patient may position the external driving magnet in the vicinity of the implant, thus forcing the driven magnet 42 away from the ridge 43 and allowing fluid from the bladder to enter the fluid pathway 20 downstream of the ridge 43, as shown in FIG. 4A.

FIG. 4B shows a valve 40 comprising a rotatable armature 46 and two driven magnets 42A, 42B. The valve 40 comprises a first driven magnet 42A attached to a first end of an armature 46 and a second driven magnet 42B with an opposing magnetic field attached to a second end of an armature 46. The driven magnets 42A, 42B are operable in a direction, X, which is substantially transverse the direction of fluid flow, Y, in the fluid pathway, as indicated in FIG. 4B.

The armature 46 is configured to rotate about a pin 47 located substantially equidistant between the first and second driven magnets 42A, 42B. The armature 46 comprises a seal 48 configured to generate a substantially fluid tight seal against an outer surface of the fluid pathway 20. More specifically, the seal 48 is configured to generate a substantially fluid tight seal against a lip 45 on the outer surface of the fluid pathway 20. Rotation of the armature 46 is caused when an external driving magnet is positioned in the vicinity of the implant 10. The external driving magnet there attracts at least one of the driven magnets 42A, 42B and repeals at least one of the driven magnets 42A, 42B, thus generating rotation of the armature 46 about the pin 47. When the valve 40 is in a closed position, the seal 48 is pressed against the lip 45, thus preventing fluid flow within the fluid pathway 20 downstream of the seal 48. Accordingly, the valve 40 comprises a bias magnet 49 configured to hold the driven magnet 42A against the lip 45. In use, a patient may position the external driving magnet in the vicinity of the implant, thus rotating the armature 46 and moving the seal 48 away from the lip 45, as shown in FIG. 4B. This enables fluid to flow from the bladder and into the fluid pathway 20 downstream of the lip 45.

In some embodiments, the valve 40 comprises at least one vent 41, as shown in FIG. 4B. The vent(s) may be configured to relieve air bubbles from within the valve 40. The vent 41 may be located adjacent to the pin 47. Alternatively, or in addition, vent 41 may be located adjacent to the second driven magnet 42B. The vent or vents 41 ensure that the moving members within the valve 40, such as the armature 46, are not subjected to any unnecessary resistance that may build up over time. The resulting valve 40 is therefore more robust.

As shown in FIGS. 7A to 7E, the implant 10 further comprises a connection mechanism 150 configured to provisionally connect the implant 10 with a deployment device 100. More specifically, the implant 10 is configured to provisionally engage with a delivery catheter 110 or a guide wire 112 thereof. The guide wire 112 comprises a connector 160 having protrusion 62. The implant 10 comprises a seat 90 having a recess 60. In some embodiments, the recess 60 is a second recess within the implant 10. The protrusion 62 is configured to provisionally connect to the recess 60. Accordingly, the connection mechanism 150 comprises the connector 160 having protrusion 62 and the seat 90 having a recess 60. Alternatively, or in addition, in some embodiments, the connector 160 comprises a protrusion and/or ridge configured to provisionally connect to a recess and/or aperture, thus provisionally connecting the implant 10 to the deployment device 100.

FIG. 5A shows a deployment device 100 for inserting the implant 10 into a patient. The deployment device 100 comprises a delivery catheter 110 having a guide wire 112 therein, wherein the guide wire 112 comprises a distal end 114 for attachment to the implant 10 and a proximal end 116 operably attached to a spring 120, as shown in FIG. 6. The deployment device 100 further comprises a trigger 130 configured to hold the spring 120 in tension and torsion and a cam 140 located between the proximal end of the guide wire 116 and the spring 120, as shown in FIG. 6.

The cam 140 is shaped to control the transmission of force between the spring 120 and the guide wire 112. More specifically, the cam 140 is shaped such that upon actuation of the trigger 130, the guide wire 112 is subjected to an axial force, followed by a rotational force, followed by an axial force.

FIG. 5B shows the deployment device 100 of FIG. 5A wherein the trigger 130 has been actuated, the expandable portion 50 if the implant has expanded and the guide wire 112 has disconnected from the implant 10. Following actuation of the trigger 130, the spring 120 is released. Initially, axial movement is transmitted to the guide wire 112, via the cam 140, thus causing the expandable portion 50 of the implant 10 to expand. Secondly, rotational movement is transmitted to the guide wire 112, via the cam 140, thus causing the guide wire connector 160 to disconnect from the seat 90 of implant 10. Finally, axial movement is again transmitted to the guide wire 112, via the cam 140, thus causing the guide wire 112 to move away from the implant 10 prior to being withdrawn from the patient's body.

FIGS. 7A to 7E show the releasable connection between the deployment device 100 and the implant 10. Initially, the guide wire 112 is operably connected to the expandable portion 50 and/or the distal end 12 of the implant 10 via a connection mechanism 150. The protrusion 62 of the connector 160 is engaged with the recess 60 of the seat 90, as shown in FIG. 7A. Consequently, when the trigger 130 is actuated the tension in the spring 120 causes the guide wire 112 and entire connection mechanism 150 to initially move axially, thus causing the expandable portion 50 of the implant 10 to expand. More specifically, the axial energy in the spring is transmitted into the guide wire 112 and subsequently into the deformable member 54 of the expandable portion 50. This causes each deformable member 54 to buckle about the groove 56 and away from the centroid 52 of the deformable portion 50.

As shown in FIG. 7B, once the deformable members 54 have deformed, the ridge 82 and first recess 80 are aligned and engaged. Next, the guide wire 112 rotates thus causing the connector 160 to rotate, as shown in FIG. 7C. Consequently, the protrusion 62 rotates and disengages with the second recess 60. The guide wire 112 and connector 160 are then free to move axially again into the position shown in FIG. 7D. Finally, the guide wire 112 and connector 160 is entirely removed from the implant 10, as shown in FIG. 7E. In FIG. 7E, the deployment device 100 has been retracted from the patient, thus leaving the implant 10 in the desired position.

FIG. 8 shows the implant seat 90 configured to provisionally connect the implant 10 to the connector 160 of guide wire 112. The seat 90 is located within the implant 10 as shown in FIG. 7E. The seat 90 is hollow. The internal surface 91 of the seat 90 forms a part of the fluid pathway 20. The seat 90 is shaped to provisionally receive the connector 160.

The seat 90 comprises a second recess 60 configured to temporarily engage with the protrusion 62 of connector 160. The temporary engagement initially causes the seat 90 to move axially and the expandable portion 50 to deform upon actuation of the trigger 130. Subsequently, when the guide wire 112 and connector 160 are rotated during actuation, the protrusion 62 is also rotated with respect to the seat 90. The protrusion 62 is rotated until it aligns with the opening 63, thus enabling the connector 160 to disengage with the seat 90. Following disengagement, the connector 160 and guide wire 112 are able to move axially once again, thus allowing the delivery catheter 110 to be withdrawn from the patient.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1: An implant for controlling fluid flow within a patient's urethra, the implant comprising:
a fluid pathway configured to receive a fluid;
a seal configured to restrict fluid flow between the fluid pathway and the patient's urethra, in use; and
a valve configured to control the flow of the fluid along the fluid pathway.

2: The implant according to claim 1, further comprising an expandable portion configured to secure the implant in a desired position, in use.

3: The implant according to claim 2, wherein the expandable portion comprises a centroid and at least two deformable members configured to deform laterally away from the centroid, in use.

4: The implant according to claim 2, wherein the expandable portion comprises at least three deformable members configured to buckle laterally away from the centroid, in use.

5: The implant according to claim 4, wherein each deformable member comprises at least one groove configured to promote lateral deformation away from the centroid, in use.

6: The implant according to claim 5, wherein each groove is located on a surface of the deformable member closest to the centroid of the expandable portion.

7: The implant according to claim 2, wherein the fluid pathway comprises an inlet positioned between the expandable portion and the seal.

8: The implant according to claim 1, wherein the seal comprises a plurality of protrusions attached to the outside of the fluid pathway, and wherein at least one of the plurality of protrusions comprises a diameter greater than the diameter of the patient's bladder neck.

9. (canceled)

10: The implant according to claim 1, wherein the implant enables a leak rate in the urethra of less than 1 ml per hour when the valve is in a closed position.

11: The implant according to claim 1, further comprising a recess configured to provisionally engage with a delivery catheter.

12: The implant according to claim 1, wherein the valve comprises at least one driven magnet configured to operate the valve between an open position and a closed position.

13: The implant according to claim 12, wherein the at least one driven magnet is operable in a direction substantially transverse to the direction of fluid flow in the fluid pathway.

14: The implant according to claim 12, wherein the at least one driven magnet is coupled to a rotatable armature comprising a seal.

15: The implant according to claim 12, wherein the valve comprises at least two opposing driven magnets coupled to a rotatable armature and wherein the armature is configured to rotate about a pin located between the at least two driven magnets.

16: The implant according to claim 15, wherein the pin is substantially equidistant between the two driven magnets.

17: The implant according to claim 12, further comprising a bias magnet configured to hold the valve in the closed position.

18: A deployment device for inserting the implant according to claim 1 into a patient, the deployment device comprising:
a delivery catheter having a guide wire therein, wherein the guide wire comprises a distal end for attachment to the implant and a proximal end operably attached to a spring; and
a trigger configured to hold the spring in tension and torsion,
wherein actuation of the trigger releases the spring, thus generating axial and rotational movement of the guide wire to disconnect the guide wire from the implant, in use.

19: The deployment device according to claim 18, further comprising a cam located between the spring and the guide wire, wherein the cam is configured to transmit axial movement from the spring to the guide wire followed by rotational movement from the spring to the guide wire followed by axial movement from the spring to the guide wire following actuation of the trigger.

20: The deployment device according to claim 18, wherein the delivery catheter comprises a protrusion configured to be provisionally received by the recess in the implant.

21: The deployment device according to claim 18, wherein the guide wire is operably attached to the distal end of the expandable portion of the implant.
